(19) United States
(12) Patent Application Publication
(10) Pub. No.: US 2007/0099918 A1
(43) Pub. Date: May 3, 2007

(54) Use of inhibitors of the EGFR-mediated signal transduction for the treatment of benign prostatic hyperplasia (BPH) / prostatic hypertrophy

(75) Inventors: Thomas Singer, Inzlingen (DE); Stefan Platz, Linkenheim-Hochstetten (DE); Florian Colbatzky, Stafflangen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/609,407

(22) Filed: Dec. 12, 2006

Related U.S. Application Data
(63) Continuation of application No. 10/431,699, filed on May 8, 2003, now abandoned.
(60) Provisional application No. 60/389,815, filed on Jun. 18, 2002.

(30) Foreign Application Priority Data
May 11, 2002 (DE) 10221018

(51) Int. Cl. A61K 31/5377; A61K 31/517

(52) U.S. Cl. 514/234.2; 514/266.4; 514/266.2; 514/266.22

(57) ABSTRACT

The present invention relates to the use of specific EGF-receptor antagonists for preparing a pharmaceutical composition for the prevention and/or treatment of benign prostatic hyperplasia and/or prostatic hypertrophy, a method for the treatment or prevention of benign prostatic hyperplasia/prostatic hypertrophy comprising administering an EGF-receptor antagonist of groups (A), (B) or (C), described herein optionally in combination with known compounds for the treatment of benign prostatic hyperplasia/prostatic hypertrophy, as well as associated pharmaceutical compositions.

### RELATED APPLICATIONS

This application claims benefit to U.S. Application Ser. No. 60/389,815 filed Jun. 18, 2003.

The present invention relates to the use of EGF-receptor antagonists which are selected from the group (A) consisting of the compounds of general formula 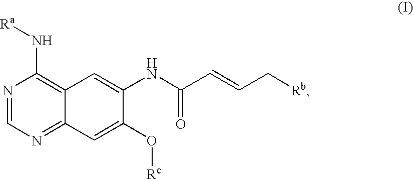
wherein R^{a}, R^{b} and R^{c} are defined as in claim 1,
the group (B) consisting of the compounds
(1) 4-[(3-chloro-4-fluorophenyl)amino]-6-[3-(morpholin-4-yl)-propyloxy]-7-methoxy-quinazoline,
(2) 4-[(3-ethynylphenyl)amino]-6,7-bis-(2-methoxy-ethoxy)-quinazoline,
(3) 4-[(3-chloro-4-fluorophenyl)amino]-7-[3-(morpholin-4-yl)-propyloxy]-6-[(vinyl-carbonyl)amino]-quinazoline,
(4) 4-[(R)-(1-phenyl-ethyl)amino]-6-(4-hydroxy-phenyl) 7H-pyrrolo[2,3-d]pyrimidine,
(5) 3-cyano-4-[(3-chloro-4-fluorophenyl)amino]-6-{[4-(N,N-dimethylamino)-1-oxo-2-buten-1-yl]amino}-7-ethoxy-quinoline or
(6) 4-{[3-chloro-4-(3-fluorobenzyloxy)-phenyl]amino}-6-(5-{[(2-methanesulphonyl-ethyl)amino]methyl}-furan-2-yl)-quinazoline and
   the group (C) consisting of the antibodies Cetuximab, Trastuzumab, ABX-EGF and Mab ICR-62,
   and, in the case of the compounds of groups (A) and (B), optionally the tautomers, the stereoisomers or the salts thereof, particularly the physiologically acceptable salts thereof with inorganic or organic acids or bases, for preparing a pharmaceutical composition for the prevention and/or treatment of benign prostatic hyperplasia (BPH) and/or prostatic hypertrophy, a method for the treatment or prevention of benign prostatic hyperplasia/prostatic hypertrophy comprising administering an EGF-receptor antagonist of groups (A), (B) or (C), optionally in combination with known compounds for the treatment of benign prostatic hyperplasia/prostatic hypertrophy, as well as pharmaceutical compositions comprising an EGF-receptor antagonist of groups (A), (B) or (C) and a known compound for the treatment of benign prostatic hyperplasia/prostatic hypertrophy, selected from the group (D) consisting of osaterone, α1-adrenoreceptor-antagonists, non-selective and selective muscarine antagonists, LHRH-/GnRH-antagonists or testosterone-5α-reductase inhibitors.

The α1-adrenoreceptor-antagonists used in the combination of active substances according to the invention are preferably the active substances KMD-3231 (silodosin), AIO-8507L, UK-338003, RBX-2258 (parvosin), SNAP-6383 (L 771688), GYKI-16084, UK-294315, (S)-doxazosin, tamsulosin, prazosin, naftopidil, terazosin, alfuzosin or indoramin.

Examples of non-selective and selective muscarine antagonists which may be used in the combination of active substances according to the invention are preferably the active substances darifenacin or tolterodine.

Examples of LHRH-/GnRH-antagonists which may be used in the combination of active substances according to the invention are preferably the active substances goserelin, desorelin, cetrorelix or gonadorelin.

Examples of testosterone-5α-reductase inhibitors which may be used in the combination of active substances according to the invention are preferably the active substances GI-198745 (dutasteride), LY-320236 (izonsteride), TF 505, AS-601811, finasteride, FK-687 or CS-891.

### BACKGROUND TO THE INVENTION

Benign prostatic hyperplasia (BPH)/prostatic hypertrophy, a benign enlargement of the prostate, is a well-known complaint in men, which generally comes to medical attention from the age of 50 onwards. About 50% of all men aged over 50 and 95% of all men aged over 70 are affected by it. Benign prostatic hyperplasia/prostatic hypertrophy is a generally progressive condition which in serious cases may endanger kidney function and require surgical intervention. The number of untreated patients runs at over 37 million worldwide. The growth of the prostate compresses or lengthens the urethra, causing the symptoms of ureteral blockage and possibly leading to urinary retention.

The prostate consists of epithelial glandular tubes embedded in fibromuscular stroma. The hyperplastic growth of the prostate begins at about the age of 30 in the periurethrally located parts of the gland, the so-called transition zone. Apart from the effects of ageing, androgenic hormones constitute a crucial stimulus to growth in the post-pubertal regulation of the volume of the gland. In the normal prostate, the enzyme 5α-reductase in the epithelial cells converts the androgenic hormone testosterone (T) into dihydrotestosterone (DHT). DHT, an active androgenic prostate metabolite, binds to cytoplasmic receptors and is transported into the cell nucleus where it initiates RNA and protein synthesis and cell replication. It is assumed that BPH occurs in response to the effects of DHT on the ageing prostate and to changes in the stroma and epithelial cells (Steers, Zorn, Dis. Mon., 41(7):437-497 (1995)).

Age-dependent changes in the serum concentrations of the hormonal regulatory circuit as a whole (LH, FSH, SHGB, T and DHT), and of other hormones which may affect this regulatory circuit (oestrogens, prolactin, testosterone derivatives), have been investigated as possible causes. However, there is no correlation between age-dependent hormonal changes in the serum and the intraprostatic hormone concentrations. Thus, it is clear that the prostate itself is responsible for regulating the hormonal milieu.

Possible points of attack for controlling the intraprostatic hormonal milieu are the 5α-reductase (i.e. the androgen metabolism), hormone receptor expression in the epithelium and stroma, oestrogens and other hormones. In addition, numerous peptidal growth factors have a paracrine or autocrine effect on the local metabolism in the various compartments of the gland, by means of which the equilibrium of the cell kinetics can be shifted between proliferation and programmed cell death.

The clinical symptoms of BPH comprise both blocking symptoms (e.g. stoppage of the stream of urine, a weak or interrupted stream, urinary retention) which result directly from the constriction of the neck of the bladder and the prostatic urethra by the hyperplastic prostate, and also symptoms of an irritated lower urinary tract (e.g. urinary frequency, nycturia, dysuria, uresiesthesis, urinary incontinence).

Untreated, BPH may lead to serious complications of the urinary tract and kidneys such as e.g. acute urinary retention and hydronephrosis (uronephrosis).

### DESCRIPTION OF THE INVENTION

An object of the present invention is to find new active substances for the prevention and/or treatment of benign prostatic hyperplasia/prostatic hypertrophy.

Surprisingly it has been found that the EGF-receptor antagonists of the abovementioned groups (A) to (C) are particularly suitable for the prevention and/or treatment of benign prostatic hyperplasia/prostatic hypertrophy.

In general formula (I) 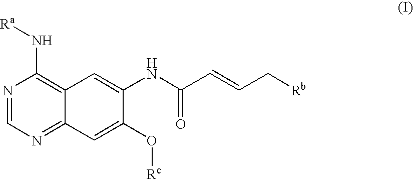
R^{a} denotes a phenyl, benzyl or 1-phenylethyl group, wherein the phenyl nucleus is substituted in each case by the groups R¹ and R², where
R¹ and R², which may be identical or different, in each case denote a hydrogen, fluorine, chlorine or bromine atom,
a methyl, trifluoromethyl, ethynyl or methoxy group,
   R^{b} denotes a di-(C₁₋₄-alkyl)-amino group wherein the alkyl moieties may be identical or different,
   an N-(C₁₋₄-alkyl)-N-(C₂₋₄-alkyl)-amino group wherein the C₂₋₄-alkyl moiety in the position β, γ or δ to the nitrogen atom of the amino group is substituted by the group R⁴, where
R⁴ denotes a C₁₋₃-alkoxy or di-(C₁₋₃-alkyl)-amino group,
a pyrrolidino, piperidino or morpholino group,
   a di-(C₂₋₄-alkyl)-amino group wherein the two C₂₋₄-alkyl moieties in the position β, γ or δ to the nitrogen atom of the amino group are each substituted by the group R⁴, while the substituents may be identical or different and R⁴ is as hereinbefore defined,
   a C₁₋₄-alkylamino group wherein the nitrogen atom is substituted by a tetrahydrofuran-3-yl, tetrahydropyran-3-yl, tetrahydropyran-4-yl, tetrahydrofuranylmethyl, 1-(C₁₋₂-alkyl)-pyrrolidin-3-yl, 1-(C₁₋₂-alkyl)-piperidin-3-yl or 1-(C₁₋₂-alkyl)-piperidin-4-yl group,
   a C₃₋₅-cycloalkylamino or C₃₋₅-cycloalkyl-C₁₋₃-alkylamino group, wherein in each case the nitrogen atom is substituted by a C₁₋₃-alkyl group,
   a 5- to 7-membered alkyleneimino group optionally substituted by 1 or 2 methyl groups which may be substituted by the group R⁴ either at a cyclic carbon atom or at one of the methyl groups, where R⁴ is as hereinbefore defined,
   a piperidino group optionally substituted by 1 or 2 methyl groups wherein the methylene group in the 4 position is replaced by an oxygen or sulphur atom, by a sulphinyl or sulphonyl group or by an imino group substituted by the group R⁵, where
R⁵ denotes a C₁₋₃-alkyl, 2-methoxyethyl, 3-methoxypropyl, C₃₋₆-cycloalkyl, C₃₋₆-cycloalkyl-C₁₋₃-alkyl, C₁₋₃-alkylcarbonyl, C₁₋₃-alkylsulphonyl, aminocarbonyl, C₁₋₃-alkylaminocarbonyl or di-(C₁₋₃-alkyl)-aminocarbonyl group, and
   R^{c} denotes a C₄₋₇-cycloalkoxy or C₃₋₇-cycloalkyl-C₁₋₄-alkoxy group, wherein the cycloalkyl moiety may be substituted in each case by a C₁₋₃-alkyl or C₁₋₃-alkoxy group, or
   a tetrahydrofuran-3-yloxy, tetrahydropyran-3-yloxy, tetrahydropyran-4-yloxy or tetrahydrofuranylmethoxy group.

Within the scope of the objects according to the invention, preferred EGF-receptor antagonists are those which are selected from the group (A′) consisting of compounds of general formula (I) wherein

R^{a} denotes a phenyl group substituted by the groups R¹ and R², where

R₁ denotes a fluorine, chlorine or bromine atom and
R₂ denotes a hydrogen or a fluorine atom,
   R^{b} denotes a di-(C₁₋₄-alkyl)-amino group wherein the alkyl moieties may be identical or different,
   a methylamino or ethylamino group, wherein in each case the nitrogen atom is substituted by a 2-methoxy-ethyl, 1-methoxy-2-propyl, 2-methoxy-propyl, 3-methoxy-propyl, tetrahydrofuran-3-yl, tetrahydropyran-4-yl, tetrahydrofuran-2-yl methyl, 1-methyl-piperidin-4-yl, 1-ethyl-piperidin-4-yl, 1-(tetrahydrofuran-3-yl)-piperidin-4-yl, cyclopropyl or cyclopropylmethyl group,
   a bis-(2-methoxyethyl)-amino group,
   a pyrrolidino, piperidino or morpholino group optionally substituted by one or two methyl groups,
   a piperazino group which is substituted in the 4 position by a methyl, ethyl, cyclopropyl, cyclopropyl methyl, 2-methoxy-ethyl, tetrahydrofuran-3-yl, tetrahydropyran-4-yl or tetrahydrofuran-2-ylmethyl group,
   a 2-(methoxymethyl)-pyrrolidino, 2-(ethoxymethyl)-pyrrolidino, 4-hydroxy-piperidino, 4-methoxy-piperidino, 4-ethoxy-piperidino, 4-(tetrahydrofuran-3-yl)-piperidino or 4-morpholino-piperidino group and
   R^{c} denotes a cyclopropylmethoxy, cyclobutylmethoxy, cyclopentylmethoxy or cyclohexylmethoxy group,
   a cyclobutyloxy, cyclopentyloxy or cyclohexyloxy group,
   a tetrahydrofuran-3-yloxy, tetrahydropyran-4-yloxy or tetrahydrofuran-2-yl methoxy group
   or are selected from the active substances of the abovementioned groups (B) and (C),
   and, in the case of the compounds of groups A′ and B, optionally the tautomers, the stereoisomers or the salts thereof.

Within the scope of the objects according to the invention, particularly preferred EGF-receptor antagonists are those which are selected from the group (A″) consisting of the compounds of general formula (I) wherein

R^{a} denotes a 3-chloro-4-fluorophenyl group,

R^{b} denotes a dimethylamino, diethylamino, bis-(2-methoxy-ethyl)-amino, N-methyl-N-(2-methoxy-ethyl)-amino, N-ethyl-N-(2-methoxy-ethyl)-amino, N-methyl-N-cyclopropyl-amino, N-methyl-N-cyclopropylmethyl-amino, N-methyl-N-(1-methoxy-2-propyl)-amino, N-methyl-N-(2-methoxy-propyl)-amino, N-methyl-N-(3-methoxy-propyl)-amino, N-methyl-N-(tetrahydrofuran-3-yl)-amino, N-methyl-N-(tetrahydropyran-4-yl)-amino, N-methyl-N-(tetrahydrofuran-2-ylmethyl)-amino or N-methyl-N-(1-methyl-piperidin-4-yl)-amino group

a pyrrolidino, piperidino or morpholino group optionally substituted by one or two methyl groups,

a piperazino group which is substituted in the 4 position by a methyl, ethyl, cyclopropylmethyl or 2-methoxy-ethyl group, and

R^{c} denotes a cyclopropylmethoxy, cyclobutyloxy or cyclopentyloxy group,

a tetrahydrofuran-3-yloxy or tetrahydrofuran-2-ylmethoxy group or are selected from the active substances of the abovementioned groups (B) and (C),

and, in the case of the compounds of groups (A″) and (B), optionally the tautomers, the stereoisomers or the salts thereof.

Within the scope of the objects according to the invention, the following compounds of the abovementioned groups (A), (B) and (C) may be used, for example:
(1) 4-[(3-chloro-4-fluorophenyl)amino]-6-{[4-(morpholin-4-yl)-1-oxo-2-buten-1-yl]amino}-7-cyclopropylmethoxy-quinazoline,
(2) 4-[(3-chloro-4-fluorophenyl)amino]-6-{[4-(N,N-diethylamino)-1-oxo-2-buten-1-yl]amino}-7-cyclopropylmethoxy-quinazoline,
(3) 4-[(3-chloro-4-fluorophenyl)amino]-6-{[4-(dimethylamino)-1-oxo-2-buten-1-yl]amino}-7-cyclopropylmethoxy-quinazoline,
(4) 4-[(3-chloro-4-fluorophenyl)amino]-6-({4-[bis-(2-methoxyethyl)-amino]-1-oxo-2-buten-1-yl}amino)-7-cyclopropylmethoxy-quinazoline,
(5) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,
(6) 4-[(3-chloro-4-fluorophenyl)amino]-6-{[4-(N,N-dimethylamino)-1-oxo-2-buten-1-yl]amino}-7-cyclopentyloxy-quinazoline,
(7) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(8) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(9) 4-[(3-chloro-4-fluorophenyl)amino]-6-({4-[N-(2-methoxy-ethyl)-N-methylamino]-1-oxo-2-buten-1-yl}amino)-7-cyclopentyloxy-quinazoline,
(10) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(11) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(12) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(13) 4-[(3-chloro-4-fluorophenyl)amino]-6-[3-(morpholin-4-yl)-propyloxy]-7-methoxy-quinazoline,
(14) 4-[(3-ethynylphenyl)amino]-6,7-bis-(2-methoxy-ethoxy)-quinazoline,
(15) 4-[(3-chloro-4-fluorophenyl)amino]-7-[3-(morpholin-4-yl)-propyloxy]-6-[(vinylcarbonyl)amino]-quinazoline,
(16) 4-[(R)-(1-phenyl-ethyl)amino]-6-(4-hydroxy-phenyl) 7H-pyrrolo[2,3-d]-pyrimidine,
(17) 3-cyano-4-[(3-chloro-4-fluorophenyl)amino]-6-{[4-(N,N-dimethylamino)-1-oxo-2-buten-1-yl]amino}-7-ethoxy-quinoline,
(18) 4-{[3-chloro-4-(3-fluorobenzyloxy)-phenyl]amino}-6-(5-{[(2-methanesulphonyl-ethyl)amino]methyl}-furan-2-yl)-quinazoline,
   the tautomers, the stereoisomers or the salts thereof,
   the antibodies Cetuximab, Trastuzumab, ABX-EGF or Mab ICR-62, while according to the invention most particular importance is attached to the compounds:
(1) 4-[(3-chloro-4-fluorophenyl)amino]-6-{[4-(morpholin-4-yl)-1-oxo-2-buten-1-yl]amino}-7-cyclopropylmethoxy-quinazoline,
(2) 4-[(3-chloro-4-fluorophenyl)amino]-6-{[4-(N,N-diethylamino)-1-oxo-2-buten-1-yl]amino}-7-cyclopropylmethoxy-quinazoline,
(3) 4-[(3-chloro-4-fluorophenyl)amino]-6-{[4-(dimethylamino)-1-oxo-2-buten-1-yl]amino}-7-cyclopropylmethoxy-quinazoline,
(4) 4-[(3-chloro-4-fluorophenyl)amino]-6-{[4-(N,N-dimethylamino)-1-oxo-2-buten-1-yl]amino}-7-cyclopentyloxy-quinazoline,
(5) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(6) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(7) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(8) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(9) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline or
(10) 4-[(3-chloro-4-fluorophenyl)amino]-6-[3-(morpholin-4-yl)-propyloxy]-7-methoxy-quinazoline,
(11) 4-[(3-ethynylphenyl)amino]-6,7-bis-(2-methoxy-ethoxy)-quinazoline,
(12) 4-[(3-chloro-4-fluorophenyl)amino]-7-[3-(morpholin-4-yl)-propyloxy]-6-[(vinylcarbonyl)amino]-quinazoline,
(13) 3-cyano-4-[(3-chloro-4-fluorophenyl)amino]-6-{[4-(N,N-dimethylamino)-1-oxo-2-buten-1-yl]amino}-7-ethoxy-quinoline,
(14) 4-{[3-chloro-4-(3-fluorobenzyloxy)-phenyl]amino}-6-(5-{[(2-methanesulphonyl-ethyl)amino]methyl}-furan-2-yl)-quinazoline,
   the tautomers, the stereoisomers or the salts thereof, but especially to the compounds:
(1) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(2) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline or
(3) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
   the tautomers, the stereoisomers or the salts thereof.

In addition, combinations of the abovementioned active substances with other substances already on the market for the treatment of benign prostatic hyperplasia/prostatic hypertrophy may also be used according to the invention.

The invention thus also relates to a method for the treatment or prevention of benign prostatic hyperplasia/prostatic hypertrophy by simultaneous or sequential administration of a therapeutically effective amount of an active substance of the abovementioned group (A), (B) or (C) in combination with a therapeutically effective amount of another medicament active against benign prostatic hyperplasia/prostatic hypertrophy (D) to a person in need of such a treatment.

The medicament (D) may be selected from the group consisting of osaterone, an α1-adrenoreceptor antagonist, non-selective and selective muscarine antagonists, LHRH/GnRH antagonists and a testosterone-5α-reductase inhibitor.

The α1-adrenoreceptor antagonist may be selected from the group consisting of the active substances KMD-3231 (selodosin), AIO-8507L, UK-338003, RBX-2258 (parvosin), SNAP-6383 (L 771688), GYKI-16084, UK-294315, (S)-doxazosin, tamsulosin, prazosin, naftopidil, terazosin, alfuzosin and indoramin.

The non-selective or selective muscarine antagonist may be selected from the group consisting of the active substances darifenacin and tolterodine.

The LHRH/GnRH antagonist may be selected from the group consisting of the active substances goserelin, desorelin, cetrorelix and gonadorelin.

The testosterone-5α-reductase inhibitor may be selected from the group consisting of the active substances GI-198745 (dutasteride), LY-320236 (izonsteride), TF 505, AS-601811, finasteride, FK-687 and CS-891.

In a preferred embodiment of the process according to the invention the medicament (D) is selected from the group consisting of tamsulosin, UK-338003, terazosin, indoramin, tolterodine, dutasteride and finasteride.

In a particularly preferred embodiment of the process according to the invention tamsulosin is used.

The dosage of components (A), (B) and (C) is 0.01 mg to 500 mg administered up to 5 times a day, preferably 0.01 mg to 300 mg up to 3 times a day.

The dosage of component (D) for the combination of pharmaceutical compositions is roughly from 1/10 of the lowest normally recommended dose to 1/1 of the normally recommended dose.

An EGF receptor antagonist of group (A), (B) or (C) as mentioned above may for example be administered in combination with

tamsulosin, which may be administered orally in a dosage of 0.2 to 0.8 mg once a day, or in combination with

naftopidil, which may be administered orally in a dosage of 25 to 100 mg per day, or in combination with

terazosin, which may be administered orally in a dosage of 1 to 20 mg per day, or in combination with

alfuzosin, which may be administered orally in a dosage of 2.5 mg three times a day to 10 mg once a day, or in combination with

indoramin, which may be administered orally in a dosage of 50 to 200 mg per day, or in combination with

doxazosin, which may be administered orally in a dosage of 1 to 16 mg per day, or in combination with

dutasteride, which may be administered orally in a dosage of 0.5 mg per day, or in combination with

finasteride, which may be administered orally in a dosage of 1 to 5 mg per day, or in combination with

tolterodine, which may be administered orally in a dosage of 2 to 4 mg per day, for the treatment of benign prostatic hyperplasia/prostatic hypertrophy.

In addition a pharmaceutical composition according to the invention may be a kit of parts for the treatment and/or prevention of benign prostatic hyperplasia/prostatic hypertrophy, this kit comprising:
(a) a first container containing a pharmaceutical composition comprising a therapeutically effect amount of an active substance selected from groups (A), (B) or (C) and one or more pharmaceutically acceptable diluents and/or carriers; and
(b) a second container containing a pharmaceutical composition comprising one of the medicaments (D) and one or more pharmaceutically acceptable diluents and/or carriers.

A preferred kit comprises tamsulosin in the second container.

In another aspect the present invention relates to the use of an active substance selected from groups (A), (B) or (C) in combination with another medicament (D) for the preparation of a pharmaceutical composition for the treatment and/or prevention of benign prostatic hyperplasia/prostatic hypertrophy.

The active substances of groups (A) to (D) which may be used for this purpose according to the invention as well as preferred embodiments can be found in the comments relating to the objects of the invention mentioned above.

The active substances may be administered orally, parenterally, by spray inhalation or rectally. Parenteral administration may consist of subcutaneous, intravenous and intramuscular injections.

For this purpose, conventional formulations may be used. For example, the active substances, optionally combined with other active substances, may be formulated with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The active substances may be administered orally in a wide variety of different dosage forms, for example they may be formulated together with different pharmaceutically acceptable inert carriers in the form of tablets, capsules, pastilles, lozenges, hard sweets, powders, atomisers, aqueous suspensions, elixirs, syrups and the like. Such carriers include for example solid diluents or fillers, sterile aqueous media and various non-toxic organic solvents. In addition, oral formulations of this kind may be suitably sweetened and/or flavoured with various agents conventionally used for this purpose. In general, the active substances are present in oral formulations of this kind at concentration levels ranging from about 0.5 wt. % to about 90 wt. %, based on the total composition, in amounts sufficient to produce the desired dosage units. Other suitable dosage forms for the active substances comprise formulations for controlled release and devices which are well known to specialists in the field.

For the purposes of parenteral administration, solutions of the active substances in sesame or groundnut oil or in aqueous propylene glycol may be used, as well as sterile aqueous solutions of the corresponding pharmaceutically acceptable salts. Such aqueous solutions should if necessary be suitably buffered and the liquid diluent made isotonic with sufficient salt or glucose. These specific aqueous solutions are particularly suitable for intravenous, intramuscular and subcutaneous injections. In connection with this, the sterile aqueous media used may easily be obtained using common methods well known in the art. For example, distilled water is normally used as the liquid diluent, and the final preparation is passed through a suitable bacterial filter such as a filter made of sintered glass or kieselguhr or unglazed porcelain. Preferred filters of this kind include the Berkefeld, Chamberland and asbestos disc metal Seitz filter, in which the fluid is sucked into a sterile container by means of a suction pump. During the preparation of these injectable solutions the necessary process steps should be taken at all times to ensure that the end products are obtained in a sterile condition. For the purposes of transdermal administration, the dosage form of the particular compound or compounds may comprise, for example, solutions, lotions, ointments, creams, gels, suppositories, formulations for sustained rate-limited release and equipment for this purpose. Such dosage forms comprise the particular compound or compounds and may contain ethanol, water, penetration promoters and inert carriers such as gel producers, mineral oil, emulsifiers, benzyl alcohol and the like.

The compounds are administered by inhalation in the form of powdered preparations with lactose and other excipients or in the form of aqueous solutions as aerosols.

The inhalable powders which may be used within the scope of the use according to the invention may contain the active substance or combination of active substances either on their own or in admixture with suitable physiologically acceptable excipients. If the active substance or combination of active substances is present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred. Lactose is particularly preferred, while lactose monohydrate is most preferred, as the excipient according to the invention.

The propellant-containing aerosols for inhalation which may be used within the scope of the use according to the invention may contain the active substance or combination of active substances dissolved in the propellent gas or in dispersed form. The propellent gases which may be used to prepare the aerosols for inhalation are known from the prior art. Suitable propellent gases are selected from among the hydrocarbons such as n-propane, n-butane or isobutane and halohydrocarbons such as preferably fluorinated derivatives of methane, ethane, propane, butane, cyclopropane or cyclobutane. The abovementioned propellent gases may be used on their own or mixed together. Particularly preferred propellent gases are fluorinated alkane derivatives selected from HFC134a (1,1,1,2-tetrafluoroethane), HFC227 (1,1,1,2,3,3,3-heptafluoropropane) and mixtures thereof.

The propellant-containing aerosols for inhalation which may be used within the scope of the use according to the invention may further contain additional ingredients such as cosolvents, stabilisers, surfactants, antioxidants, lubricants and pH adjusters. All these ingredients are known in the art.

If the active substance or combination of active substances according to the invention is administered by inhalation in the form of propellant-free solutions or suspensions, aqueous or alcoholic, preferably ethanolic solutions may be used as solvent. The solvent may be exclusively water or it may be a mixture of water and ethanol. The relative proportion of ethanol to water is not restricted, but the maximum limit is preferably up to 70 percent by volume, particularly up to 60 percent by volume and most preferably up to 30 percent by volume. Solutions or suspensions containing the active substance or combination of active substances are optionally adjusted with suitable acids to a pH of 2 to 7, preferably 2 to 5. This pH may be adjusted using acids selected from inorganic or organic acids. Examples of particularly suitable inorganic acids are hydrochloric acid, hydrobromic acid, nitric acid, sulphuric acid and/or phosphoric acid. Examples of particularly suitable organic acids are: ascorbic acid, citric acid, malic acid, tartaric acid, maleic acid, succinic acid, fumaric acid, acetic acid, formic acid and/or propionic acid and others. Preferred inorganic acids are hydrochloric acid and sulphuric acid. Of the organic acids, ascorbic acid, fumaric acid and citric acid are preferred. If desired, mixtures of the abovementioned acids may also be used, particularly in the case of acids which have other properties, in addition to their acidifying properties, e.g. as flavourings, antioxidants or complexing agents, such as for example citric acid or ascorbic acid. According to the invention, hydrochloric acid is most preferably used to adjust the pH.

The abovementioned compounds and their salts have valuable properties. They are particularly suitable for the prevention and/or treatment of benign prostatic hyperplasia/prostatic hypertrophy.

For example the following compounds
compound a=4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
compound b=4-[(3-chloro-4-fluorophenyl)amino]-6-{[4-(N-cyclopropyl-N-methyl-amino)-1-oxo-2-buten-1-yl]amino}-7-cyclopentyloxy-quinazoline and
compound c=4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
   were tested for their prostate-reducing activity.
   Experimental Section
   1) Test Method:

Three groups of five male rats (about 7 weeks old and weighing about 185 g) are treated for 14-15 days with an EGF-R-TK inhibitor. The active substance is administered once a day as a suspension through an oesophageal tube. After the test period has ended the animals are killed, their prostates are removed and weighed.

The results of the series of tests are listed in the Tables that follow.

2) Test Results:

TABLE 1
Administration of compound a (length of treatment: 14 days)

| | Dosage group | | | |
|---|---|---|---|---|
| | control | compound a | | |
| | Dosage level | | | |
| | 0 mg/kg | 10 mg/kg | 30 mg/kg | 100 mg/kg |
| average absolute weight | 0.4641 | 0.4100 | 0.3419* | 0.1099* |
| [g] | | | | |
| average relative weight | 0.1913 | 0.1718 | 0.1473* | 0.0672* |
| [% of body weight] | | | | |
| average relative weight | 23.64 | 20.62 | 17.07* | 5.55* |
| [% of the brain] | | | | |

*p < 0.05

TABLE 2
Administration of compound b (length of treatment: 14 days)

| | Dosage group | | | |
|---|---|---|---|---|
| | control | compound b | | |
| | Dosage level | | | |
| | 0 mg/kg | 10 mg/kg | 30 mg/kg | 100 mg/kg |
| average absolute weight | 0.4641 | 0.3983* | 0.3989 | 0.1848* |
| [g] | | | | |
| average relative weight | 0.1913 | 0.1622* | 0.1697 | 0.1191* |
| [% of body weight] | | | | |
| average relative weight | 23.64 | 20.23* | 20.71* | 10.13* |
| [% of the brain] | | | | |

*p < 0.05

TABLE 3
Administration of compound c (length of treatment: 15 days)

| | Dosage group | | | |
|---|---|---|---|---|
| | control | compound c | | |
| | Dosage level | | | |
| | 0 mg/kg | 10 mg/kg | 30 mg/kg | 100 mg/kg |
| average absolute weight | 0.4641 | 0.3854* | 0.2242* | 0.1463* |
| [g] | | | | |
| average relative weight | 0.1913 | 0.1681 | 0.1117* | 0.0892* |
| [% of body weight] | | | | |
| average relative weight | 23.64 | 19.67* | 11.50* | 7.63* |
| [% of the brain] | | | | |

*p < 0.05

The abovementioned compounds, the preparation of which has not already been described in the prior art, are obtained by the following method:

### EXAMPLE 1

### 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline

4.70 ml of oxalyl chloride are added dropwise to a solution of 4.50 g of bromocrotonic acid in 60 ml of methylene chloride. Then one drop of N,N-dimethylformamide is added. After about 30 minutes the development of gas has ceased and the reaction mixture is evaporated down in the rotary evaporator. The crude bromocrotonic acid chloride is taken up in 30 ml of methylene chloride and, while cooling with an ice bath, added dropwise to a solution of 7.00 g of 4-[(3-chloro-4-fluorophenyl)amino]-6-amino-7-cyclopropylmethoxy-quinazoline and 10.20 ml of Hünig base in 150 ml of tetrahydrofuran. The reaction mixture is stirred for about 1.5 hours while cooling with an ice bath and for a further two hours at ambient temperature. Then 5.20 g of N-(2-methoxy-ethyl)-N-methylamine are added and the reaction mixture is stirred overnight at ambient temperature. For working up it is diluted with methylene chloride and washed thoroughly with water. The organic phase is dried over magnesium sulphate and evaporated down. The crude product is purified by chromatography over a silica gel column with ethyl acetate followed by ethyl acetate/methanol (19:1) as eluant.

Yield: 5.07 g (51% of theory)

Mass spectrum (ESI⁻): m/z=512, 514 [M−H]⁻

R_{f} value: 0.25 (silica gel, ethyl acetate/methanol=9:1)

The following compounds are obtained analogously to Example 1:
(1) 4-[(3-chloro-4-fluorophenyl)amino]-6-{[4-(N,N-dimethylamino)-1-oxo-2-buten-1-yl]amino}-7-cyclopentyloxy-quinazoline,
(2) 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline,
(3) 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,
(4) 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline,
(5) 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline,
(6) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(7) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(8) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,
(9) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(10) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(11) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline.

We claim:

1. A method of treating benign prostatic hyperplasia/prostatic hypertrophy which comprises administering to a patient in need thereof a therapeutically effective amount of EGF-receptor antagonists selected from the group (A) consisting of compounds of general formula 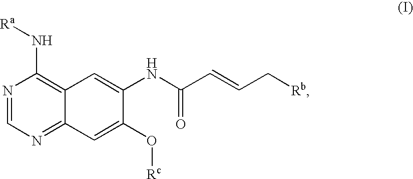
wherein
R^{a} denotes a phenyl, benzyl or 1-phenylethyl group, wherein the phenyl nucleus is substituted in each case by the groups R¹ and R², while
R¹ and R², which may be identical or different, in each case denote a hydrogen, fluorine, chlorine or bromine atom,
a methyl, trifluoromethyl, ethynyl or methoxy group,
R^{b} denotes a di-(C₁₋₄-alkyl)-amino group wherein the alkyl moieties may be identical or different,
an N—(C₁₋₄-alkyl)-N-(C₂₋₄-alkyl)-amino group wherein the C₂₋₄-alkyl moiety in the position β, γ or δ to the nitrogen atom of the amino group is substituted by the group R⁴, where
R⁴ denotes a C₁₋₃-alkoxy or di-(C₁₋₃-alkyl)-amino group,
a pyrrolidino, piperidino or morpholino group,
a di-(C₂₋₄-alkyl)-amino group wherein the two C₂₋₄-alkyl moieties in the position β, γ or δ to the nitrogen atom of the amino group are each substituted by the group R⁴, while the substituents may be identical or different and R⁴ is as hereinbefore defined,
a C₁₋₄-alkylamino group wherein the nitrogen atom is substituted by a tetrahydrofuran-3-yl, tetrahydropyran-3-yl, tetrahydropyran-4-yl, tetrahydrofuranylmethyl, 1-(C₁₋₂-alkyl)-pyrrolidin-3-yl, 1-(C₁₋₂-alkyl)-piperidin-3-yl or 1-(C₁₋₂-alkyl)-piperidin-4-yl group,
a C₃₋₅-cycloalkylamino or C₃₋₅-cycloalkyl-C₁₋₃-alkylamino group, wherein in each case the nitrogen atom is substituted by a C₁₋₃-alkyl group,
a 5- to 7-membered alkyleneimino group optionally substituted by 1 or 2 methyl groups which may be substituted by the group R⁴ either at a cyclic carbon atom or at one of the methyl groups, where R⁴ is as hereinbefore defined,
a piperidino group optionally substituted by 1 or 2 methyl groups wherein the methylene group in the 4 position is replaced by an oxygen or sulphur atom, by a sulphinyl or sulphonyl group or by an imino group substituted by the group R⁵, while
R⁵ denotes a C₁₋₃-alkyl, 2-methoxyethyl, 3-methoxypropyl, C₃₋₆-cycloalkyl, C₃₋₆-cycloalkyl-C₁₋₃-alkyl, C₁₋₃-alkylcarbonyl, C₁₋₃-alkylsulphonyl, aminocarbonyl, C₁₋₃-alkylaminocarbonyl or di-(C₁₋₃-alkyl)-aminocarbonyl group, and
R^{c} denotes a C₄₋₇-cycloalkoxy or C₃₋₇-cycloalkyl-C₁₋₄-alkoxy group, wherein the cycloalkyl moiety may be substituted in each case by a C₁₋₃-alkyl or C₁₋₃-alkoxy group, or
a tetrahydrofuran-3-yloxy, tetrahydropyran-3-yloxy, tetrahydropyran-4-yloxy or tetrahydrofuranylmethoxy group,
a C₂₋₄-alkoxy group which may be substituted in the β, γ or δ position to the oxygen atom by an azetidin-1-yl, 4-methyl-homopiperazino or 4-ethyl-homopiperazino group,
the group (B) consisting of the compounds
(1) 4-[(3-chloro-4-fluorophenyl)amino]-6-[3-(morpholin-4-yl)-propyloxy]-7-methoxy-quinazoline,
(2) 4-[(3-ethynylphenyl)amino]-6,7-bis-(2-methoxy-ethoxy)-quinazoline,
(3) 4-[(3-chloro-4-fluorophenyl)amino]-7-[3-(morpholin-4-yl)-propyloxy]-6-[(vinyl-carbonyl)amino]-quinazoline,
(4) 4-[(R)-(1-phenyl-ethyl)amino]-6-(4-hydroxy-phenyl) 7H-pyrrolo[2,3-d]pyrimidine,
(5) 3-cyano-4-[(3-chloro-4-fluorophenyl)amino]-6-{[4-(N,N-dimethylamino)-1-oxo-2-buten-1-yl]amino}-7-ethoxy-quinoline or
(6) 4-{[3-chloro-4-(3-fluorobenzyloxy)-phenyl]amino}-6-(5-{[(2-methanesulphonyl-ethyl)amino]methyl}-furan-2-yl)-quinazoline,
the tautomers, the stereoisomers or the salts thereof and
the group (C) consisting of the antibodies Cetuximab, Trastuzumab, ABX-EGF and Mab ICR-62 for preparing a pharmaceutical composition for the prevention of benign prostatic hyperplasia (BPH) and/or prostatic hypertrophy.

2. Method according to claim 1, wherein the EGF-receptor antagonists of group (A) are selected from the group (A′) consisting of the compounds of general formula (I) and wherein
R^{a} denotes a phenyl group substituted by the groups R¹ and R², where
R₁ denotes a fluorine, chlorine or bromine atom and
R₂ denotes a hydrogen or a fluorine atom,
R^{b} denotes a di-(C₁₋₄-alkyl)-amino group wherein the alkyl moieties may be identical or different,
a methylamino or ethylamino group, wherein in each case the nitrogen atom is substituted by a 2-methoxy-ethyl, 1-methoxy-2-propyl, 2-methoxy-propyl, 3-methoxy-propyl, tetrahydrofuran-3-yl, tetrahydropyran-4-yl, tetrahydrofuran-2-yl methyl, 1-methyl-piperidin-4-yl, 1-ethyl-piperidin-4-yl, 1-(tetrahydrofuran-3-yl)-piperidin-4-yl, cyclopropyl or cyclopropylmethyl group,
a bis-(2-methoxyethyl)-amino group,
a pyrrolidino, piperidino or morpholino group optionally substituted by one or two methyl groups,
a piperazino group which is substituted in the 4 position by a methyl, ethyl, cyclopropyl, cyclopropyl methyl, 2-methoxy-ethyl, tetrahydrofuran-3-yl, tetrahydropyran-4-yl or tetrahydrofuran-2-ylmethyl group,
a 2-(methoxymethyl)-pyrrolidino, 2-(ethoxymethyl)-pyrrolidino, 4-hydroxy-piperidino, 4-methoxy-piperidino, 4-ethoxy-piperidino, 4-(tetrahydrofuran-3-yl)-piperidino or 4-morpholino-piperidino group and
R^{c} denotes a cyclopropylmethoxy, cyclobutylmethoxy, cyclopentylmethoxy or cyclohexylmethoxy group,
a cyclobutyloxy, cyclopentyloxy or cyclohexyloxy group,
a tetrahydrofuran-3-yloxy, tetrahydropyran-4-yloxy or tetrahydrofuran-2-yl methoxy group,
the tautomers, stereoisomers and salts thereof.

3. Method according to claim 1, wherein in that the EGF-receptor antagonists of group (A) are selected from the group (A″) consisting of compounds of general formula I, wherein
R^{a} denotes a 3-chloro-4-fluorophenyl group,
R^{b} denotes a dimethylamino, diethylamino, bis-(2-methoxy-ethyl)-amino, N-methyl-N-(2-methoxy-ethyl)-amino, N-ethyl-N-(2-methoxy-ethyl)-amino, N-methyl-N-cyclopropyl-amino, N-methyl-N-cyclopropylmethyl-amino, N-methyl-N-(1-methoxy--propyl)-amino, N-methyl-N-(2-methoxy-propyl)-amino, N-methyl-N-(3-methoxy-propyl)-amino, N-methyl-N-(tetrahydrofuran-3-yl)-amino, N-methyl-N-(tetrahydropyran-4-yl)-amino, N-methyl-N-(tetrahydrofuran-2-ylmethyl)-amino or N-methyl-N-(1-methyl-piperidin-4-yl)-amino group
a pyrrolidino, piperidino or morpholino group optionally substituted by one or two methyl groups,
a piperazino group which is substituted in the 4 position by a methyl, ethyl, cyclopropylmethyl or 2-methoxy-ethyl group, and
R^{c} denotes a cyclopropylmethoxy, cyclobutyloxy or cyclopentyloxy group,
a tetrahydrofuran-3-yloxy or tetrahydrofuran-2-ylmethoxy group,
the tautomers, stereoisomers and salts thereof.

4. The method of claim 1, wherein the EFG receptor antagonist is selected from the group consisting of
(1) 4-[(3-chloro-4-fluorophenyl)amino]-6-{[4-(morpholin-4-yl)-1-oxo-2-buten-1-yl]amino}-7-cyclopropylmethoxy-quinazoline,
(2) 4-[(3-chloro-4-fluorophenyl)amino]-6-{[4-(N,N-diethylamino)-1-oxo-2-buten-1-yl]amino}-7-cyclopropylmethoxy-quinazoline,
(3) 4-[(3-chloro-4-fluorophenyl)amino]-6-{[4-(dimethylamino)-1-oxo-2-buten-1-yl]amino}-7-cyclopropylmethoxy-quinazoline,
(4) 4-[(3-chloro-4-fluorophenyl)amino]-6-({4-[bis-(2-methoxyethyl)-amino]-oxo-2-buten-1-yl}amino)-7-cyclopropylmethoxy-quinazoline,
(5) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,
(6) 4-[(3-chloro-4-fluorophenyl)amino]-6-{[4-(N,N-dimethylamino)-1-oxo-2-buten-1-yl]amino}-7-cyclopentyloxy-quinazoline,
(7) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(8) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(9) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,
(10) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(11) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(12) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(13) 4-[(3-chloro-4-fluorophenyl)amino]-6-[3-(morpholin-4-yl)-propyloxy]-7-methoxy-quinazoline,
(14) 4-[(3-ethynylphenyl)amino]-6,7-bis-(2-methoxy-ethoxy)-quinazoline,
(15) 4-[(3-chloro-4-fluorophenyl)amino]-7-[3-(morpholin-4-yl)-propyloxy]-6-[(vinylcarbonyl)amino]-quinazoline,
(16) 4-[(R)-(1-phenyl-ethyl)amino]-6-(4-hydroxy-phenyl) 7H-pyrrolo[2,3-d]-pyrimidine,
(17) 3-cyano-4-[(3-chloro-4-fluorophenyl)amino]-6-{[4-(N,N-dimethylamino)-1-oxo-2-buten-1-yl]amino}-7-ethoxy-quinoline,
(18) 4-{[3-chloro-4-(3-fluorobenzyloxy)-phenyl]amino}-6-(5-{[(2-methanesulphonyl-ethyl)amino]methyl}-furan-2-yl)-quinazoline,
the tautomers, stereoisomers and salts thereof,
the antibodies Cetuximab, Trastuzumab, ABX-EGF and Mab ICR-62.

5. The method of claim 1, wherein the EGF-receptor antagonists are selected from the group consisting of
(1) 4-[(3-chloro-4-fluorophenyl)amino]-6-{[4-(morpholin-4-yl)-1-oxo-2-buten-1-yl]amino}-7-cyclopropylmethoxy-quinazoline,
(2) 4-[(3-chloro-4-fluorophenyl)amino]-6-{[4-(N,N-diethylamino)-1-oxo-2-buten-1-yl]amino}-7-cyclopropylmethoxy-quinazoline,
(3) 4-[(3-chloro-4-fluorophenyl)amino]-6-{[4-(dimethylamino)-1-oxo-2-buten-1-yl]amino}-7-cyclopropylmethoxy-quinazoline,
(4) 4-[(3-chloro-4-fluorophenyl)amino]-6-{[4-(N,N-dimethylamino)-1-oxo-2-buten-1-yl]amino}-7-cyclopentyloxy-quinazoline,
(5) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(6) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(7) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(8) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(9) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline or
(10) 4-[(3-chloro-4-fluorophenyl)amino]-6-[3-(morpholin-4-yl)-propyloxy]-7-methoxy-quinazoline,
(11) 4-[(3-ethynylphenyl)amino]-6,7-bis-(2-methoxy-ethoxy)-quinazoline,
(12) 4-[(3-chloro-4-fluorophenyl)amino]-7-[3-(morpholin-4-yl)-propyloxy]-6-[(vinylcarbonyl)amino]-quinazoline,
(13) 3-cyano-4-[(3-chloro-4-fluorophenyl)amino]-6-{[4-(N,N-dimethylamino)-1-oxo-2-buten-1-yl]amino}-7-ethoxy-quinoline,
(14) 4-{[3-chloro-4-(3-fluorobenzyloxy)-phenyl]amino}-6-(5-{[(2-methanesulphonyl-ethyl)amino]methyl}-furan-2-yl)-quinazoline,
the tautomers, stereoisomers and salts thereof.

6. The method of claim 1, wherein the EGF-receptor antagonist is selected from the group consisting of
(1) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(2) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(3) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
the tautomers, stereoisomers and salts thereof.

7. Method for the prevention and/or treatment of benign prostatic hyperplasia/prostatic hypertrophy by simultaneous or sequential administration of an active substance of groups (A), (B) or (C) according to claim 1 in combination with a therapeutically effective amount of a further medicament active against benign prostatic hyperplasia/prostatic hypertrophy (D), selected from the group consisting of osaterone, α1-adrenoreceptor antagonists, non-selective and selective muscarine antagonists, LHRH/GnRH antagonists and testosterone-5α-reductase inhibitors, to a person requiring such treatment.

8. Method according to claim 7, wherein the α1-adrenoreceptor antagonist is selected from the group consisting of the active substances KMD-3231 (silodosin), AIO-8507L, UK-338003, RBX-2258 (parvosin), SNAP-6383 (L 771688), GYKI-16084, UK-294315, (S)-doxazosin, tamsulosin, prazosin, naftopidil, terazosin, alfuzosin and indoramin.

9. Method according to claim 7, wherein the non-selective or selective muscarine antagonist is selected from the group consisting of the active substances darifenacin and tolterodine.

10. Method according to claim 7, wherein the LHRH/GnRH antagonist is selected from the group consisting of the active substances goserelin, desorelin, cetrorelix and gonadorelin.

11. Method according to claim 7, wherein the testosterone-5α-reductase inhibitor is selected from the group consisting of the active substances GI-198745 (dutasteride), LY-320236 (izonsteride), TF 505, AS-601811, finasteride, FK-687 and CS-891.

12. Method according to claim 7, wherein the medicament (D) is selected from the group consisting of tamsulosin, UK-338003, terazosin, indoramin, tolterodine, dutasteride and finasteride.

13. Method according to claim 7, characterised in that the medicament (D) is tamsulosin.

14. Kit of parts for the treatment and/or prevention of benign prostatic hyperplasia/prostatic hypertrophy, this kit comprising:
(a) a first container containing a pharmaceutical composition comprising a therapeutically effect amount of an active substance selected from groups (A), (B) or (C) of claim 7 and one or more pharmaceutically acceptable diluents and/or carriers; and
(b) a second container containing a pharmaceutical composition comprising one of the medicaments (D) of claim 7 and one or more pharmaceutically acceptable diluents and/or carriers.

15. Kit of parts according to claim 14, wherein the medicament (D) is tamsulosin.

16. Use of an active substance selected from the groups (A), (B) and (C) of claim 2 in combination with a further medicament (D) of claim 7 for preparing a pharmaceutical composition for the treatment of benign prostatic hyperplasia/prostatic hypertrophy.

17. Use of an active substance selected from the groups (A), (B) and (C) of claim 7 in combination with a further medicament (D) of claim 7 for preparing a pharmaceutical composition for the prevention of benign prostatic hyperplasia/prostatic hypertrophy.

18. A pharmaceutical composition comprised of an active substance selected from the groups (A), (B) and (C) of claim 7 in combination with a further medicament of claim 7.
